(19) United States
(12) Patent Application Publication
(10) Pub. No.: US 2007/0239039 A1
(43) Pub. Date: Oct. 11, 2007

(54) Method and apparatus for measuring blood pressures by using blood oxygen concentration and electrocardiography

(75) Inventors: Paul Yang, Taipei (TW); Jen-Chien Chien, Yilan County (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD., CHUNG HO CITY (TW)

(21) Appl. No.: 11/398,679

(22) Filed: Apr. 6, 2006

(51) Int. Cl. A61B 5/02

(52) U.S. Cl. 600/483

(57) ABSTRACT

The present invention discloses a method and an apparatus for measuring blood pressures by using blood oxygen concentration and electrocardiography, and the method uses a blood oxygen concentration analyzer to measure a continuous change of waveforms of a blood oxygen cycle during a cardio arterial pulse and an electrocardiography to confirm the pulse period of the systolic pressure and diastolic pressure of the blood pressure and correspond the pulse period with the continuous change of waveforms measured by the blood oxygen concentration analyzer. The microprocessor computes the corresponding cross-sectional areas of the continuous change of waveforms and the pulse period measured by the electrocardiogram, and calculates the values of the systolic pressure and diastolic pressure by a preinstalled formula, so as to accurately measure the values of blood pressures (systolic pressure and diastolic pressure).

### BACKGROUND OF THE INVENTION

1. Field of the Invention

The present invention relates to a method and an apparatus for measuring blood pressures by using blood oxygen concentration and electrocardiography simultaneously, and the measurement is a non-penetrating measurement that uses the continuous change of waveforms of the blood oxygen cycle measured by a blood oxygen concentration analyzer and a systolic pressure and a diastolic pressure of the pulse period measured by an electrocardiogram to obtain accurate blood pressures.

2. Description of the Related Art

Sphygmomanometers are divided into two main types: penetrating and non-penetrating, and the non-penetrating sphygmomanometers are divided into traditional mercury sphygmomanometers and electronic sphygmomanometers, and most of the electronic sphygmomanometers sold in the market mainly adopt an auscultatory method or a resonance method, and the auscultatory method of the electronic sphygmomanometers adopts the Korotkoff sound technique to determine the systolic pressure and the diastolic pressure. The cuff of the sphygmomanometer is wound around an arm or a wrist first, and then the cuff is inflated and pressurized until the arteries of the arm or wrist completely stop the blood flow, so that the arteries under the cuff no longer pulse, and then the air in the cuff is released slowly. When the pressure is released, the pulse in the blood vessel is detected constantly. Before the pressure in the cuff approaches the systolic pressure, the pressure detector will sense the pulse of the arteries under the cuff. If the Korotkoff sound I is detected, the reading on the sphygmomanometer will be taken, and such reading is the systolic pressure (SP). Later, the pressure in the cuff is unloaded gradually, and thus the passage of the blood vessel becomes larger, the time taken becomes longer, and the time for the blood passing through the blood vessel becomes longer, until the pressure of the cuff drops to a certain level. By then, the passage of the blood vessel becomes larger, and the pressure jet effect disappears gradually. If the Korotkoff sound starts inactivating, the last sound is recorded, and such reading is the diastolic pressure (DP).

Referring to FIG. 1, the continuous curved waveform a shows the change of blood pressures in a blood vessel, and the oblique line b shows the values of pressure change when the air is released from a cuff. The first intersection point of the oblique line b at the upper left side and the continuous curved waveform a is the systolic pressure obtained by the Korotkoff sound technique, and the last intersection point of the oblique line b at the lower right side and the continuous curved waveform a is the diastolic pressure obtained by the Korotkoff sound technique, and several intersection points in the middle are the sounds of blood turbulence of the Korotkoff sounds. Since the cuff is inflated or deflated for a certain period of time, therefore there is a time gap and it is unable to obtain the actual values of the blood pressures. In the process of inflating or deflating the cuff, a user's arm or wrist will be pressed. Further, it takes some time for measuring the blood pressure, users will feel very uncomfortable.

In view of the foregoing shortcomings of the prior art, some manufacturers use a signal change of the capacity of the blood vessel to find the accurate blood pressures. Since the change of signals for the capacity of blood vessel and the blood pressure are physical magnitudes produced by the same circulation system, and the change of signals for the capacity of the blood vessel is the periodic change of the blood quantity per unit area in the blood vessel caused by the pulse cycle of the heart, and the diameter of the blood vessel will be changed by the work done by the pressure, and such change of diameter of the blood vessel will be affected by the continuous blood pressure to become continuous. At present, a common method is to use an optical detection which is divided into a reflective method and a penetrating method. The reflective optical sensor detects a light energy emitted by the LED at the derma, and the penetrating optical sensor uses two LED lights of different wavelengths (such as red light and infrared light) to penetrate through a finger tissue, and a receiver under the finger will receive the signals, and the change of the diameter of the blood vessel caused by the pulse will create a deviation of the projected light, and the angle of deviation can be plotted as a continuous waveform by converting light energy into electric energy. In FIG. 1A, the blood pressure and the change of capacity of the blood vessel produce the same periodic signal waves similarly, and a cycle of blood pressure is defined as starting from the beginning of a heart contraction to the beginning of the next heart contraction, which is a period between wave peaks of a signal for each cycle. Since the optical measurement reflects the change of capacity of the blood vessel and does not show the absolute value of the capacity of the blood vessel, therefore the amplitude of the waveform with the change of measured diameter of the blood vessel on the axis of time simply indicates the potential of the retrieved data. Since a convolution should have been carried out for the results of the heart contraction and the blood vessel wall action and the positions of the wave peak and the wave trough of each cycle of the blood pressure cannot be calculated accurately, therefore the values of blood pressure cannot be found accurately.

### SUMMARY OF THE INVENTION

In view of the shortcomings of the prior art, the inventor of the invention based on years of experience in the related industry to conduct extensive researches and experiments, and finally invented a method and an apparatus for measuring blood pressures by using blood oxygen concentration and electrocardiography.

Therefore, it is a primary objective of the present invention to provide a method for measuring blood pressures by blood oxygen concentration and electrocardiography, and the method uses a blood oxygen concentration analyzer to measure the continuous change of waveforms of a blood oxygen cycle and an electrocardiography to measure the change of potentials of systolic and diastolic states of the heart, so as to measure the blood pressures accurately. The method comprises the steps of measuring the oxygen concentration in the blood by a blood oxygen concentration analyzer to plot the continuous periodic change of waveforms caused by the systolic and diastolic states of the heart, while using an electrocardiogram to measure the potential change of the systolic and diastolic states of the heart at the same period, and using a microprocessor to compute the cross-sectional areas of the continuous periodic change of waveforms of the blood oxygen concentration, and using the measurement of the electrocardiogram to confirm the time for calculating the systolic pressure and the diastolic pressure, and after the readings are converted and processed by a preinstalled formula of the microprocessor, accurate values of the systolic pressure and the diastolic pressure can be obtained. Thus, users not only measure an accurate blood pressure, but also measure the pulse beat per minute, the oxygen content in the blood, the electrocardiogram and waveform of the blood pressure.

Another objective of the present invention is to provide an apparatus for measuring blood pressures by blood oxygen concentration and electrocardiography and the apparatus comprises a body, a display screen disposed on the surface of the body for displaying results, two corresponding electrodes disposed on the surface of the body for clamping a user's finger, a probe disposed at a side of the body, and a microprocessor and a storage device installed in the body and connected to a series of circuits for processing and storing related data respectively, so that when the apparatus is used, a user's thumb presses on the electrode while the probe clamps one of the user's fingers to obtain the waveform of a periodic continuous change of blood oxygen cycle and the period of the systolic and diastolic states of the heart, and the display screen clearly shows the alternate displays of the two. After the microprocessor processes the data, the values of the systolic pressure and the diastolic pressure can be found.

A further objective of the present invention is to provide an apparatus for measuring blood pressures by blood oxygen concentration and electrocardiography, and the apparatus comprises a body, a display disposed on a surface of the body for displaying results, an electric circuit coupled to the body, and two corresponding electrodes coupled to another end of the electric circuit for attaching a human body, a probe disposed on a side of the body, and a microprocessor and a storage device connected to a series of circuits for processing and storing related data respectively, so that when the apparatus is used, the electrodes are pressed onto a wrist of a user's body while the probe clamps one of the user's fingers to obtain the waveform of a periodic continuous change of blood oxygen cycle and the period of the systolic and diastolic states of the heart, and the display screen clearly shows the alternate display of the two. After the microprocessor processes the data, the values of the systolic pressure and the diastolic pressure can be found, and such arrangement allows users to monitor their blood pressure 24 hours a day.

Another further objective of the present invention is to provide an apparatus for measuring blood pressures by blood oxygen concentration and electrocardiography, and the apparatus comprises a body, a microprocessor and a storage device installed in the body and connected to a series of related circuit, and the microprocessor is connected to an input/output device for carrying out a cable or a wireless signal transmission.

### BRIEF DESCRIPTION OF THE DRAWINGS

The present invention will now be described in more detail hereinafter with reference to the accompanying drawings that show various embodiments of the invention, in which:

FIG. 1 is a schematic view of the measuring results of a prior art blood pressure measurement method;

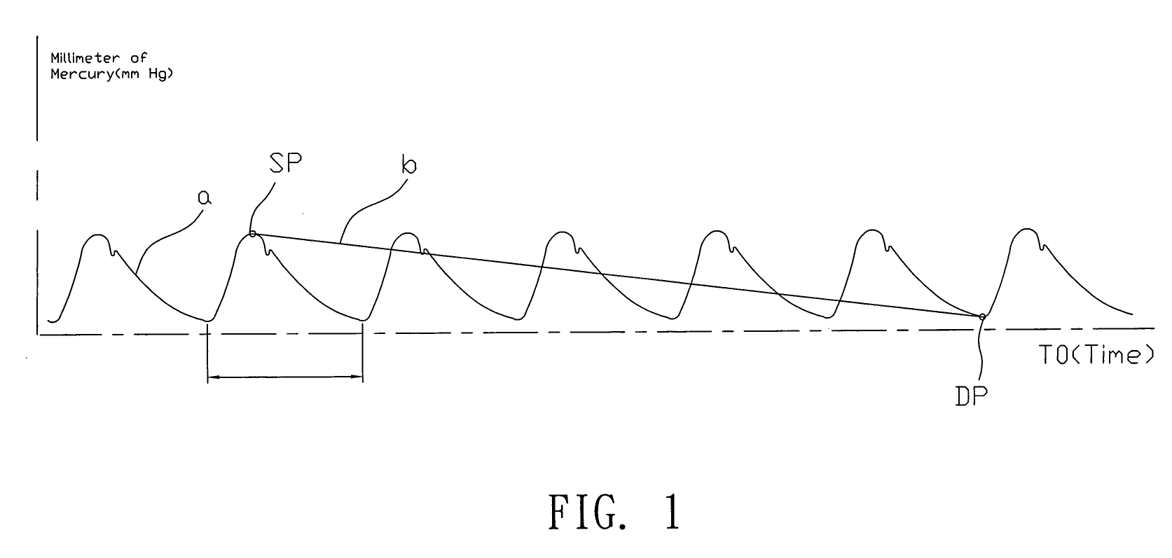

FIG. 1A is a schematic view of the measuring results of another prior art blood pressure measurement method;

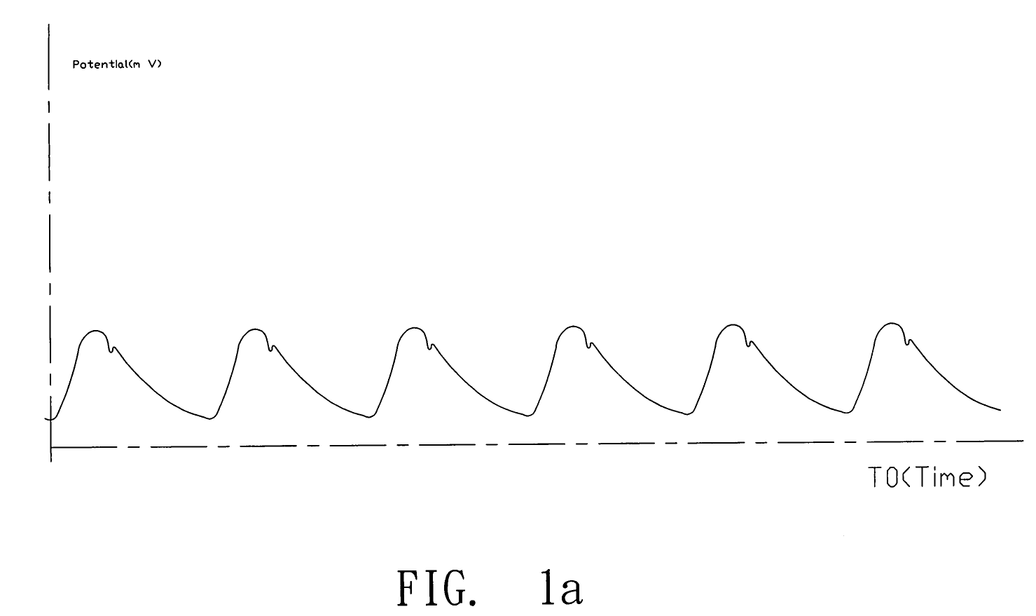

FIG. 2 is a schematic view of the waveforms measured by the present invention;

FIG. 2A is an enlarged view of the waveforms measured by the present invention;

FIG. 3 is a schematic circuit block diagram of the present invention;

FIG. 4 is a schematic view of the apparatus of the present invention;

FIG. 5 is a schematic view of the application of the present invention; and

FIG. 6 is a schematic view of another application of the present invention.

### DETAILED DESCRIPTION OF THE PREFERRED EMBODIMENTS

The present invention relates to a method for measuring blood pressures by blood oxygen concentration and electrocardiography, and the blood oxygen concentration refers to the percentage of the capacity of oxyhemoglobin (HbO₂) that combines oxygen in the blood occupied in the total capacity of the combined hemoglobin (HB). In other words, the measurement of a blood oxygen concentration (SPO₂) adopting the pulse for measuring the blood oxygen concentration includes two sections: the measurement obtained by a spectrophotometer and the plotting of blood capacity. In this preferred embodiment, the measurements obtained by the spectrophotometer and the blood oxygen concentration analyzer can be substituted by the measurements obtained by other methods adopted by those skilled in the art.

The blood oxygen concentration analyzer uses a spectrophotometer with the wavelength of a red light 660 nm and an infrared light 940 nm, and the oxyhemoglobin (HbO₂) has a higher absorption of the 660 nm red light and a lower absorption of the 940 nm infrared light. On the contrary, the hemoglobin (HB) has a lower absorption of the 660 nm and a higher absorption of the 940 nm infrared light. By the ratio of the infrared light absorption and the red light absorption of the spectrophotometer, the oxygen concentration of the hemoglobin can be obtained.

When the blood oxygen concentration analyzer measures the blood oxygen concentration in a blood, the value of the blood oxygen concentration can be measured. In addition, the light energy is projected onto the peripheral tissues of the blood vessels, and the deterioration level of the light energy is related to the heartbeat due to the blood in the blood vessel being pulsed by the systolic and diastolic states of the heart. If the heart contracts, the capacity of blood around the periphery of the blood vessel is the largest, and thus absorbing most light energy, and the light absorption is the largest, and the detected light energy is the smallest. If the heart expands, the conditions will be the opposite. Therefore, the change of the light energy absorption reflects the change of the capacity of the blood vessel. By the method of converting light energy into electric energy, the change of the capacity of the blood vessel on the axis of time can plot a continuous change of waveforms in a blood oxygen cycle.

Further, the signals of the electrocardiography (ECG) measures the change of the potentials of the systolic and diastolic state of the heart, and the cardio muscle is the only muscle in a human body that can pulsate by itself and contract with a rhythm, and the heart system sends out electric waves to stimulate the muscle tissues to contract, and thus the generation and conduction of electric waves will drive the muscle to produce a feeble current all over the body. If the electrodes of the signals of the electrocardiogram are connected to different parts of the body, we can plot the electrocardiogram.

The principle is to use the weak current produced by the systolic and diastolic movements of the heart. If such current flows through the whole body (human body is a conductor), the electrodes placed on the foot can be transferred to an ammeter and the waveform can be recorded on a paper which is the electrocardiogram.

At present, a common clinical way is the 12-process electrocardiography (ECG) that include three standard processes I, II, III, three pressurizing processes aVR, aVL, aVF, and six chest processes V1, V2, V3, V4, V5, V6. As the heart contracts, a series of changes of the potential along the time axis occur, and these changes involves an auricle depolarization, an auricle repolarization, a ventricle depolarization, a ventricle repolarization in sequence, and the waves produced include a P wave, a QRS wave, and a T wave. The physiological significance of each waveform are as follows: the P stands for the auricle depolarization, the QRS wave stands for the ventricle depolarization, the T wave stands for the ventricle repolarization repolarization, and the PR period stands for the time between the left and right auricles depolarization, and the depolarization wave sent to the auricles and ventricles, and the QT period stands for the time between the left and right ventricles depolarization and repolarization, and the PR period of the heartbeat waveform is the systolic period of the heart, and the PST period is the diastolic period of the heart.

Basically, the preferred embodiment of the invention uses a first process for the measurement to illustrate the relation between the electrocardiography and the present invention, but persons in the art can use other methods for the measurement. The method adopted by this preferred embodiment requests users to touch two different electrodes by both hands, and measures the slight change of current produced by the heartbeat, so as to plot the systolic and diastolic period of the heart on the axis of time of the electrocardiogram.

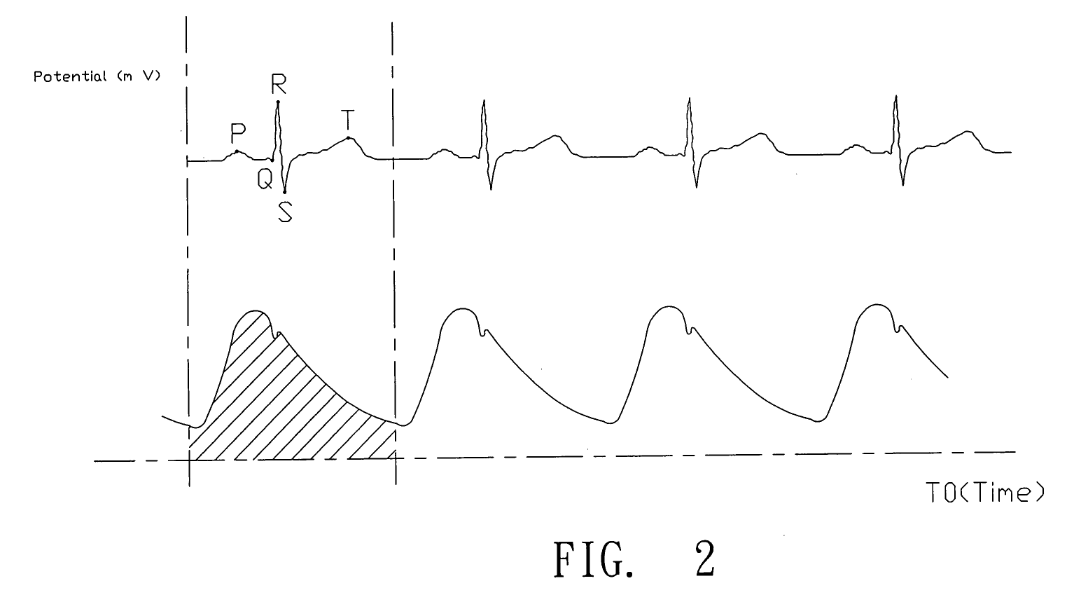

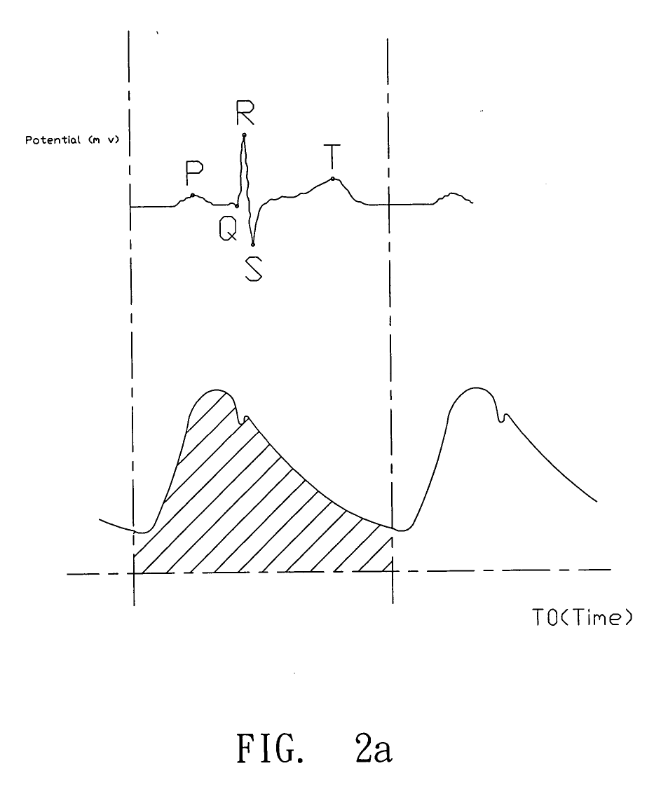

Referring to FIGS. 2 and 2A, the method of the present invention uses the electrocardiogram analyzer to confirm the computing periods for the systolic pressure and the diastolic pressure, and converts the potential of the continuous change of the blood oxygen cycle measured by the blood oxygen concentration into the corresponding potential of the heartbeat of the electrocardiogram on the same time axis. The potential waveform converted from the continuous change of the blood oxygen cycle at the axis of time corresponds to a cross-sectional area of the PR period of the waveform of the heartbeat on the electrocardiogram and also corresponds to the cross-sectional area of the PST period of the waveform of the heartbeat on the electrocardiogram. After the microprocessor processes the data by a preinstalled formula, the values of the systolic pressure and the diastolic pressure can be obtained. Thus, users not only accurately measure the systolic/diastolic pressure, but also measure the number of pulse/minute, the oxygen content in the blood, the electrocardiogram, and the waveform of the blood pressure.

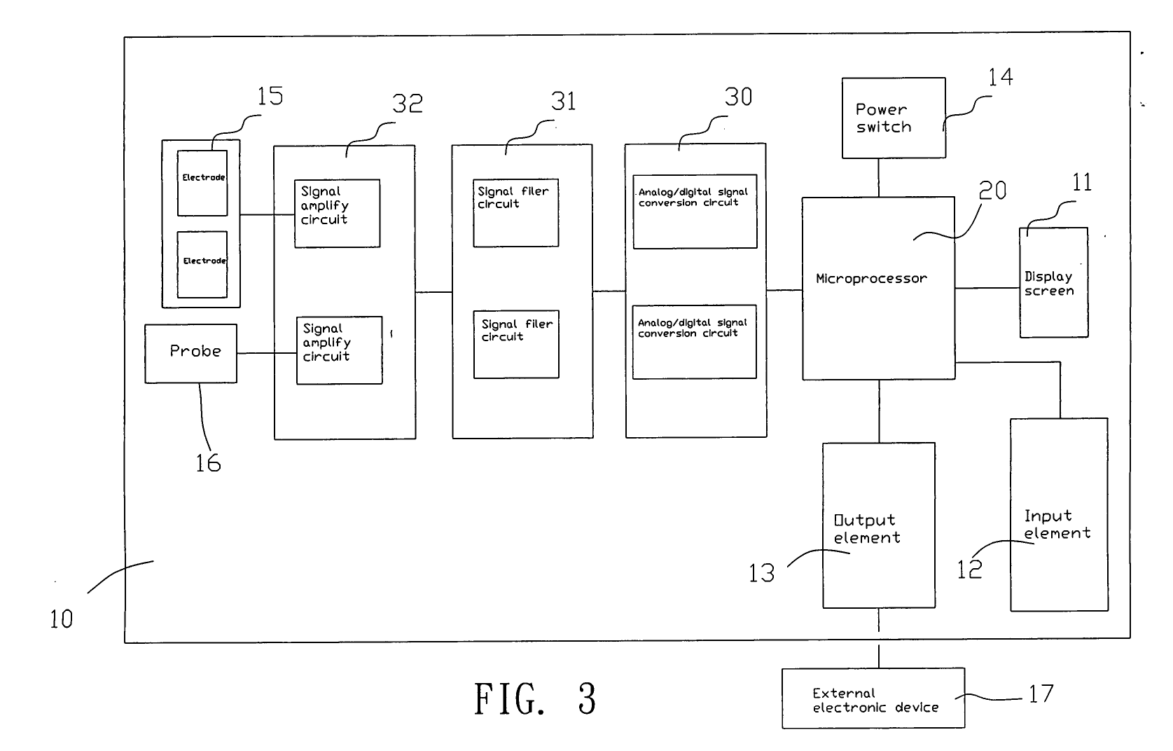

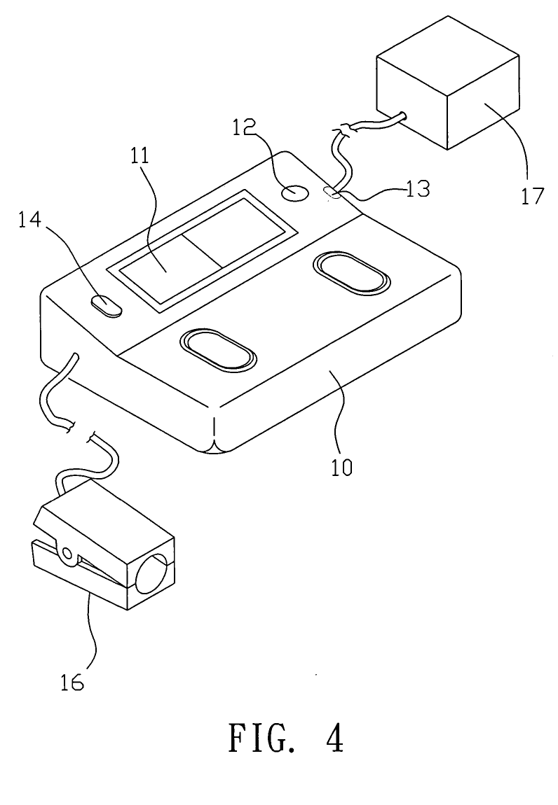

Referring to FIGS. 3 and 4 and the foregoing method, the apparatus comprises a body 10, a microprocessor 20 installed on the body 10 and coupled separately to a display screen 11, input and output elements 12, 13, and a power switch 14. The display screen 11 is installed on the surface of the body 10 for displaying the measured results, and the input element 12 is installed on the surface of the body 10 for entering related configurations and data (such as displaying the values of the systolic/diastolic pressure, electrocardiogram, or blood pressure waveform), and the output element 13 could be a cable or a wireless transmission device that is coupled to other external electronic device 17 (such as PDA and computer, etc) and the microprocessor 20 stores related data. Further, the microprocessor 20 is connected in series with an analog/digital signal conversion circuit 30, a signal filer circuit 31 and a signal amplify circuit 32, wherein the signal amplify circuit 32 is coupled with the electrode 15 and the probe 16 on the surface of the body 1, and two fluorescent lamps are installed on one side of the probe 16 and one emits red light while the other emits infrared light, and a receiver is installed on another side of the probe 16 for detecting the signals of the potential converted from the red light and infrared light of the arteries of the finger. Since skin, muscle, fat, vein blood, pigment and bone have constant absorption of these two kinds of lights and only the HBO₂ and HB concentrations of the blood will change according to the periodic change of the blood in the arteries, therefore the intensity of the signal outputted from the optoelectronic sensor will be changed periodically, and the signals of these periodic changes are processed to obtain the corresponding blood oxygen concentration. In the meantime, the pulse rate can be computed, and the continuous change of waveform of the blood oxygen cycle can be plotted, and the electrodes are used to measure the change of potentials of the systolic and diastolic changes of the heart.

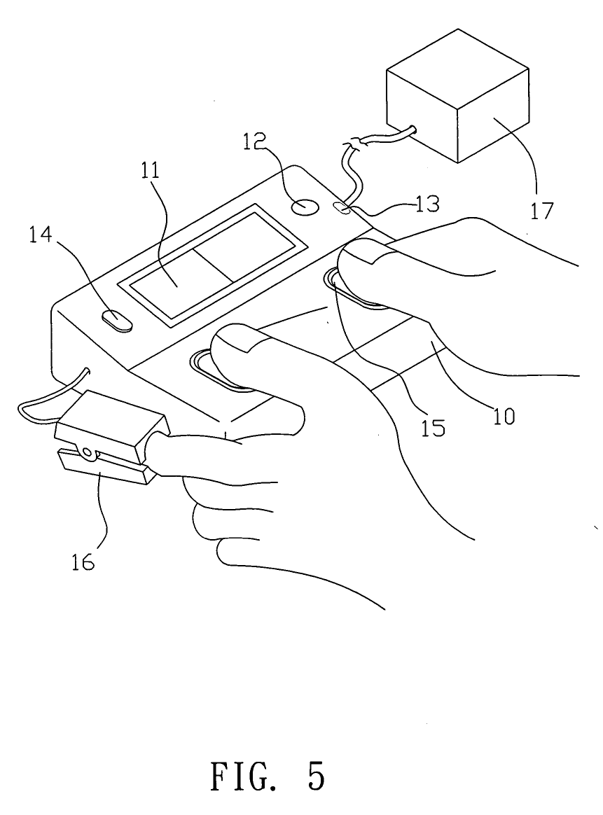

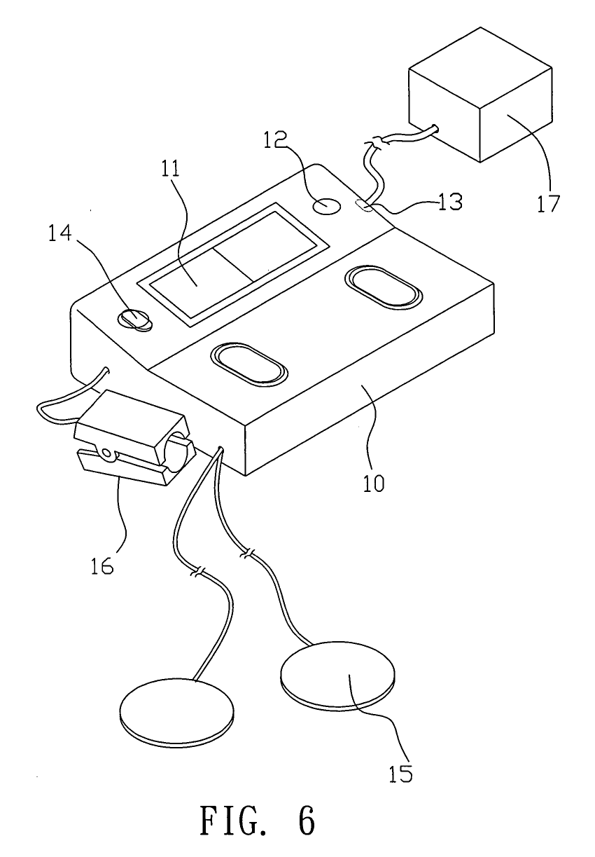

When the apparatus is used, a user's thumb presses onto an electrode 15 as shown in FIG. 5, or the electrodes 15 are extended out form the body 10 by an electric circuit and attached on a corresponding position of the user's body as shown in FIG. 6. In the meantime, the probe 16 clamps one of the user's finger other than the thumb, and the continuous change of waveforms of the blood oxygen cycle and the systolic and diastolic periods of the heart can be found. By the display screen 11, users can see the change of the two clearly. Through the conversion made by an analog/digital signal conversion circuit 30, a signal filer circuit 31 and a signal amplify circuit 32, the data is sent to the microprocessor 20. After the data processing by a formula preinstalled in the microprocessor 20, the accurate values of the systolic pressure and diastolic pressure can be found.

In summation of the description above, the method of measuring blood pressures by using blood oxygen concentration and electrocardiography, herein enhances the performance than the conventional structure and further complies with the patent application requirements.

While the invention has been described by way of examples and in terms of preferred embodiments, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method for measuring blood pressures by blood oxygen concentration and electrocardiography, comprising the steps of: using a blood oxygen concentration analyzer to measure a continuous change of waveforms of a blood oxygen cycle of a heart pulse along the same axis of time, while using an electrocardiogram to measure corresponding systolic and diastolic periods of a heart; using a microprocessor to compute cross-sectional areas on an axis of time corresponding to said continuous change of waveforms of said blood oxygen cycle; using the measurement of said electrocardiogram to confirm the period of a systolic pressure and a diastolic pressure; and obtaining the values of said systolic pressure and said diastolic pressure after said microprocessor processes said values by a preinstalled formula.

2. The method for measuring blood pressures by blood oxygen concentration and electrocardiography of claim 1, further comprising the step of calculating the average of the cross-sectional areas of the cycles of said continuous change of waveforms of said blood oxygen cycles computed by at least two continuous systolic/diastolic periods.

3. An apparatus for measuring blood pressures by blood oxygen concentration and electrocardiography, and said apparatus comprising:
a microprocessor, for controlling, computing and processing data;
a display screen, coupled with said microprocessor for displaying results;
an input element, coupled with said microprocessor for inputting related data;
an analog/digital signal conversion circuit, coupled with said microprocessor for converting an analog signal into a digital signal;
a signal filer circuit, coupled with said analog/digital signal conversion circuit for filter noses;
a signal amplify circuit, coupled with said signal filer circuit for amplifying signals;
two electrodes, coupled to said signal amplify circuit for contacting a human body for a measurement; and
a probe, coupled with said signal amplify circuit and provided for said analog/digital signal conversion circuit for a measurement.

4. The apparatus for measuring blood pressures by blood oxygen concentration and electrocardiography of claim 3, wherein said apparatus is integrated into a whole machine.

5. The apparatus for measuring blood pressures by blood oxygen concentration and electrocardiography of claim 3, wherein said microprocessor is coupled to a wireless or cable output element.

6. The apparatus for measuring blood pressures by blood oxygen concentration and electrocardiography of claim 3, wherein said electrodes are disposed on a body of said apparatus for clamping a user's finger.

7. The apparatus for measuring blood pressures by blood oxygen concentration and electrocardiography of claim 3, wherein said electrodes are extended out of said body of said apparatus by an electric circuit and attached onto a user's body.
